(19) United States
(12) Patent Application Publication
(10) Pub. No.: US 2011/0053183 A1
(43) Pub. Date: Mar. 3, 2011

(54) METHOD OF CONCENTRATING HUMAN MESENCHYMAL STEM CELLS

(76) Inventors: Yumi Matsuzaki, Tokyo (JP); Yo Mabuchi, Tokyo (JP); Satoru Morikawa, Tokyo (JP); Hideyuki Okano, Tokyo (JP)

(21) Appl. No.: 12/676,827

(22) PCT Filed: Sep. 8, 2008
(86) PCT No.: PCT/JP2008/066169

§ 371 Date: Jul. 13, 2010

(30) Foreign Application Priority Data
Sep. 6, 2007 (JP) 2007-231298

(51) Int. Cl. G01N 33/53; C12N 5/077; C12N 5/078

(52) U.S. Cl. 435/7.21; 435/366; 435/372

(57) ABSTRACT

The present invention is intended to provide methods for highly enriching human mesenchymal stem cells from a cell population containing the human mesenchymal stem cells. To highly enrich human mesenchymal stem cells, CD271⁺CD90⁺ cells are recovered by using flow cytometry etc. from a cell population containing the human mesenchymal stem cells. If the cell population contains blood cells (as in the case of a cell population prepared from a bone marrow, a peripheral blood etc.), CD45⁻CD235a⁻CD271⁺CD90⁺ cells are recovered. These cell fractions contain with high purity the mesenchymal stem cells having self-renewal capability, self-replicating capability and pluripotency. Therefore, human mesenchymal stem cells can be highly enriched by recovering CD271⁺CD90⁺ cells from the cell population containing the human mesenchymal stem cells.

### TECHNICAL FIELD

The present invention relates to methods for enriching human mesenchymal stem cells by using cell surface antigens.

### BACKGROUND ART

Mesenchymal stem cells possess self-renewal capability as well as pluripotency to differentiate into mesenchymal cells such as osteoblasts, bone cells, adipocytes, chondrocytes, myocytes, stroma cells and tendon cells, and therefore they are expected to be applicable in regenerative medicine for bones, cartilages, muscles, and the like.

The mesenchymal stem cells have been isolated by growing cells derived from a tissue such as a bone marrow, which have been cultured for a long time and attached to the culture dish. Therefore, the differentiation capability of the mesenchymal stem cells thus obtained could vary depending on different conditions for the culture, insufficient proficiency of the experimenter, different methods to be employed, and the like. This has been causing serious problems in controlling the purity and quality of the mesenchymal stem cells.

Accordingly, several methods have been developed to isolate mesenchymal stem cells by using surface antigen markers . So far, CD10, CD13, CD73 (ecto-5′ nucleotidase, SH3, SH4), CD105 (endoglin, SH2), CD166 (ALCAM) etc. have been identified as positive markers for mesenchymal stem cells, whereas CD34, CD45 etc. have been identified as negative markers. More recently, CD271 (LNGFR), CD140b (PDGFR-β), CD340 (HER-2/erbB2), CD349 (frizzled-9) etc. are also used (Buhring Hans-Jorg, et al., “Novel markers for the prospective isolation of human MSC”, Annals of the New York Academy of Sciences, annals-1392-000, Haematopoietic Stem Cells VI, 10 Nov. 2006.). Still, they are not sufficient to obtain highly pure and homogeneous mesenchymal stem cells.

### SUMMARY OF INVENTION

### Technical Problem

Under these circumstances, development of methods for isolating purer and more homogeneous mesenchymal stem cells possessing all of the self-replicating capability, self-renewal capability and pluripotency has been expected.

Accordingly, the present invention is intended to provide methods for highly enriching human mesenchymal stem cells from a cell population containing human mesenchymal stem cells, as well as kits to be used therein.

### Solution to Problem

As described in the undermentioned examples, the inventors of the present invention have recovered and analyzed the CD45⁻CD235a⁻CD271⁺CD90⁺ cells, which are not expressing CD45 nor CD235a but are expressing CD271 and CD90, isolated by a flow cytometry from a cell population contained in a human bone marrow, and discovered this cell fraction contained highly pure mesenchymal stem cells that possess high CFU-F (fibroblast colony forming unit) activity as well as the capability to differentiate into osteoblasts, chondrocytes, adipocytes etc., and thus achieved the present invention.

Accordingly, a method for enriching human mesenchymal stem cells according to the present invention includes the step of selecting CD271⁺CD90⁺ cells expressing CD271 (LNGFR) and CD90 (Thy-1) from a cell population containing the human mesenchymal stem cells. In this method, the CD271⁺CD90⁺ cells may be selected by using an anti-CD271 (LNGFR) antibody and an anti-CD90 (Thy-1) antibody. The method may include the step of preparing the cell population from a bone marrow, or the step of preparing the cell population from a peripheral blood after administration of G-CSF. The step of preparing the cell population may include the step of treating the bone marrow by collagenase.

The method for enriching human mesenchymal stem cells according to the present invention may also include the step of selecting CD45⁻CD235a⁻ cells that are not expressing CD45 nor CD235a. In this method, the CD45⁻CD235a⁻ cells may be selected by using an anti-CD45 antibody and an anti-CD235a antibody.

In the method for enriching human mesenchymal stem cells according to the present invention, the cells may be selected by using flow cytometry.

A kit according to the present invention includes an anti-CD271 antibody and an anti-CD90 antibody. The kit may also include an anti-CD45 antibody and an anti-CD235a antibody. The kit may also include collagenase.

It should be noted that “enriching (specific) cells” as used herein means to increase the ratio of the specific cells among a cell population.

### BRIEF DESCRIPTION OF DRAWINGS

FIG. 1 shows results obtained by analysis of the reactivity of human myelocytes with PI, an anti-CD45 antibody, an anti-CD235a antibody, an anti-CD271 antibody and an anti-CD90 antibody using flow cytometry in one example of the present invention.

FIG. 2 shows an effect of collagenase treatment on the recovery rate of the mesenchymal stem cells recovered from human bone marrow in one example of the present invention.

FIG. 3 shows results of analysis of the pluripotency of the CD45⁻CD235a⁻CD271⁺CD90⁺ cells recovered by using flow cytometry in one example of the present invention.

FIG. 4 shows results of analysis using flow cytometry, which indicate that the CD45⁻CD235a⁻CD271⁺CD90⁺ cells are present in tissues other than the bone marrow in one example of the present invention.

### DESCRIPTION OF EMBODIMENTS

Hereinafter the embodiments of the present invention are described more specifically and in detail by giving examples, which should not be construed as limiting the present invention.

When using a commercial kit or a measuring instrument, protocols attached to them are used unless otherwise noted.

The object, characteristics, and advantages of the present invention as well as the idea thereof will be apparent to those skilled in the art from the descriptions given herein, and the present invention can be easily reproduced by those skilled in the art based on the descriptions given herein. It is to be understood that the embodiments and specific examples of the invention described herein are to be taken as preferred examples of the present invention. These descriptions are only for illustrative and explanatory purposes and are not intended to limit the invention to these embodiments or examples. It is further apparent to those skilled in the art that various changes and modifications may be made based on the descriptions given herein within the intent and scope of the present invention disclosed herein.

### (1) Method for Enriching Human Mesenchymal Stem Cells

As used herein, mesenchymal stem cell means a cell that possesses the CFU-F (fibroblast colony forming unit) activity as well as the pluripotency to differentiate into osteoblasts, chondrocytes, and adipocytes. It should be noted that the mesenchymal stem cell could differentiate also into chondrocytes, myocytes, stroma cells, tendon cells and the like, depending on the condition for inducing differentiation.

The inventors of the present invention have made it possible to highly enrich mesenchymal stem cells by selecting a fraction of CD271⁺CD90⁺ cells from a cell population containing the human mesenchymal stem cells. If blood cells are contained in the cell population containing the human mesenchymal stem cells, the method may also include the step of selecting CD45⁻CD235a⁻ cells in order to select non-blood cells.

The specific methods for enriching human mesenchymal stem cells are hereinafter explained.

The method for enriching human mesenchymal stem cells according to the present invention includes the steps of preparing a cell population and selecting the human mesenchymal stem cells.

### (i) The Step of Preparing a Cell Population

In this step, a cell population containing human mesenchymal stem cells is prepared by flow cytometry, or affinity chromatography. It is preferable that the cells in the cell population are dissociated into individual cells and unnecessary cells are removed at this preparation step, because the cells are subsequently subjected to selection on the basis of expression of surface antigens.

While the material from which the cell population is obtained is not particularly limited, bone marrow and peripheral blood (including the peripheral blood after an administration of G-CSF) are exemplified. The bone marrow that derived from a spine, a sternum, an ilium or the like may be used.

In preparing the cell population of interest from such a material, when the material is a cluster of cells containing mesenchymal stem cells like the bone marrow, it may be treated in order to dissociate the contained cells by physical treatment such as pipetting or by chemical treatment such as enzyme digestion. As for the enzyme, any enzyme commonly used such as trypsin and collagenase may be used, but treatment using the collagenase is preferred. In a case where the cells are not completely dissociated into individual cells but some cell clusters remain even after the treatment for dissociation, it is preferable to remove the cell clusters by using a mesh etc.

If some erythrocytes are contaminated in the material in such a case as obtaining the cell population of interest from peripheral blood, it is preferable to hemolyse them in advance. The method therefor is not particularly limited, but the material may be treated in a hypotonic solution (such as water).

The cell population containing human mesenchymal stem cells is thus prepared by applying an appropriate treatment depending on the material to be used.

### (ii) The Step of Selecting Human Mesenchymal Stem Cells

In this step, the cell population prepared in “(i) The step of preparing a cell population” is used to select CD271⁺CD90⁺ cells alive.

The method for selecting CD271⁺CD90⁺ cells is not particularly limited. For example, since CD271 (LNGFR) is a receptor which binds with ligand such as neurotrophins (NGF, BDGF, NT-3 and NT-4), CD271⁺ cells can be selected by an affinity chromatography utilizing a protein obtained by an in vitro expression and purification of either of the ligands. However, in view of simplicity, the methods utilizing antibodies as described below are preferable.

The antibodies to be used in this step are an anti-CD271 antibody and an anti-CD90 antibody that are capable of selecting CD271⁺CD90⁺ cells. For example, if the flow cytometry is used, live cells can be quickly selected by using a combination of an anti-CD271 antibody and an anti-CD90 antibody which are labeled with different fluorescent dyes such as FITC, PE, APC etc. Other than the flow cytometry, CD271⁺CD90⁺ cells can be selected alive by various methods such as those using magnetic beads or those using affinity chromatography. The type of the antibody (a monoclonal antibody or a polyclonal antibody; IgG or IgM; a whole antibody molecule or an Fab fragment; etc), as well as the concentration of the antibody, may be appropriately selected by the user depending on the type of the cell population, the activity of the antibody, the method to used the antibody, and the like.

It should be noted that prior to employing any of the abovementioned methods, dead cells may be removed by allowing the cell population to react with a fluorescent dye to stain dead cells such as PI (propidium iodide) and then removing fluorescence-labeled cells.

In the case that the cell population includes blood cells, the method according to the present invention preferably includes the step of selecting CD45⁻CD235a⁻ cells. The method for this selection is not particularly limited. Similarly to the above, CD45⁻CD235a⁻ cells can be selected from the cell population by the flow cytometry utilizing fluorescence-labeled antibodies, as well as by the methods utilizing magnetic beads or affinity chromatography. The selection of CD45⁻CD235a⁻ cells may be conducted before, after, or at the same time of the selection of CD271⁺CD90⁺ cells.

The CD271+CD90⁺ cells are thus selected from the cell population containing the human mesenchymal stem cells.

### (2) Usefulness of the Method for Enriching Human Mesenchymal Stem Cells According to the Present Invention

Currently in the fields of regenerative medicine, where a tissue to be transplanted is provided by another individual (a donor), shortage of donors as well as rejections against the transplanted tissues are causing problems. In contrast, the method for enriching human mesenchymal stem cells according to the present invention can highly enrich the mesenchymal stem cells which are derived from the tissue of the subject himself, such as bone marrow, peripheral blood, or peripheral blood after administration of G-CSF. Therefore, by using the method for enriching human mesenchymal stem cells according to the present invention, the mesenchymal stem cells of the subject himself can be selected efficiently from a small amount of the tissue of the subject himself. The cells thus obtained can be autotransplanted to a desired site for differentiation into osteoblasts, bone cells, adipocytes, chondrocytes, myocytes, stroma cells, tendon cells or the like, thereby allowing regeneration of the desired cell or tissue efficiently, as well as solving the problems of the shortage of donors, the rejections, etc.

### (3) Kit

A kit for easily enriching human mesenchymal stem cells in accordance with the method of the present invention may include an anti-CD271 antibody and an anti-CD90 antibody. If blood cells should be removed while enriching the human mesenchymal stem cells, the kit may also include an anti-CD45 antibody and an anti-CD235a antibody. Further, for efficient preparation of a cell population from a desired material, the kit may also include an enzyme such as collagenase. Commercially available antibodies may be used, or a new antibody may be prepared by any technique known to those skilled in the art.

### EXAMPLES

Hereinafter embodiments of the present invention as explained above are specifically described by giving examples, which should be construed as being presented for only illustrative purpose but not to limit the present invention.

### Example 1

### Selection of CD45⁻CD235a⁻CD271⁺CD90⁺ Cells

### (1) Preparation of Cell Populations

Human costal pieces left over in respiratory surgeries were used as a starting material. When the material was insufficient, bone marrows purchased from Cambrex (Cat. Nos. 2M-125C and 2M-125D) were also used.

First, costal pieces (1 cm×1 cm) were washed with PBS, chopped by using surgical scissors, and suspended in HBSS⁺ (calcium- and magnesium-free Hanks-balanced salt solution supplemented with 2% FCS, 10 mM HEPES, and 1% penicillin/streptomycin), from which liquid phase was removed by aspiration.

The remaining bony pieces were further shredded by the scissors, placed in 0.2% collagenase solution (Wako 032-10534) in 10 mM HEPES with 1% P/S and incubated at 37° C. for 1 hour on a shaker. As control experiments, the same procedures were conducted without the 0.2% collagenase solution using the remaining bony pieces.

Finally, the collagenase-treated samples were filtered through cell strainers (Falcon 2350) to remove debris of bones. The cell suspensions thus obtained were centrifuged (×1200 rpm) at 4° C. for 7 min. For removal of erythrocytes contaminated in the cell populations, the pellets after the centrifugation were added with 1 ml of water (Sigma W3500) and agitated for 5 to 10 seconds, then resuspended in the Rescue solution (4% FBS, 2×PBS, Sigma D1408). These samples were filtered again through the cell strainers to remove debris of the erythrocytes, and the suspensions of human myelocytes were thus obtained.

The bone marrows purchased from Cambrex were kept frozen in liquid nitrogen until thawed prior to each experiment. First, a HBSS⁺ solution supplemented with DNaseI (hereinafter referred to as DNaseI HBSS⁺ solution) was warmed in a constant temperature bath at 37° C. The vials containing the frozen bone marrow (2M-125C or 2M-125D) were placed in the 37° C. bath to quickly thaw them until a small frozen piece remains (for 1 to 2 min). The myelocytes were suspended in the DNaseI HBSS⁺ solution and transferred to 15 ml centrifuge tubes. After addition of DNaseI HBSS⁺ solution up to a total volume of 10 ml, they were centrifuged (×1200 rpm) at room temperature for 7 min. Supernatants after the centrifugation were removed by gentle pipetting not to disturb the cell pellets, which were then resuspended in 1 ml of fresh DNaseI HBSS⁺ solution to obtain the suspensions of the human myelocytes.

(2) Reaction with Antibodies

The human myelocyte suspension obtained by the method described above was diluted in HBSS⁺ to the concentration of 2.5 to 5×10⁷ cells/ml.

Then, 2.5 to 5×10⁷ cells of the human myelocytes, 50 μl of an undiluted FITC-labeled anti-human CD45 antibody (DAKO), 50 μl of an undiluted FITC-labeled anti-human CD235a (Glycophorin A) antibody (DAKO), 50 μl of an undiluted PE-labeled anti-human CD271 (low-affinity nerve growth factor receptor) antibody (Miltenyi Biotec), and 50 μl of an undiluted APC-labeled anti-CD90 (Thy-1) antibody (BD Biosciences Pharmingen) were added, and incubated on ice for 30 min.

After the reaction, 10 ml of HBSS+ was added to the cell suspension and centrifuged (×1200 rpm) at 4° C. for 7 min. After the supernatant was discarded, HBSS+ containing 2 μg/ml propidium iodide (PI) (Sigma Chemical Co.) was added to the resultant pellet and the cells were suspended at a concentration of 1×10⁷ cell/ml. The suspension was filtered by using a sterile nylon-mesh filter of 60 mm or less (Miltenyi Biotec) to remove cell clusters, and the cell suspension thus obtained was used in the following analysis utilizing the flow cytometry (FACS analysis).

In the control experiments for examining non-specific binding of the abovementioned antibodies to the cells, an FITC-labeled anti-MouseIgG1 kappa antibody (eBioscience), a PE-labeled anti-Mouse IgG1 antibody (eBioscience), and an APC-labeled anti-MouseIgG1 kappa antibody (eBioscience) were used.

### (3) Fractionation of Human Mesenchymal Stem Cells

The myelocytes reacted with the antibodies were fractioned by using FACS on the basis of the reactivity of each antibody. For the FACS, MoFlo and FACS Vantage, equipped with an argon laser at 488 nm and a RED laser at 600 to 650 nm, were used.

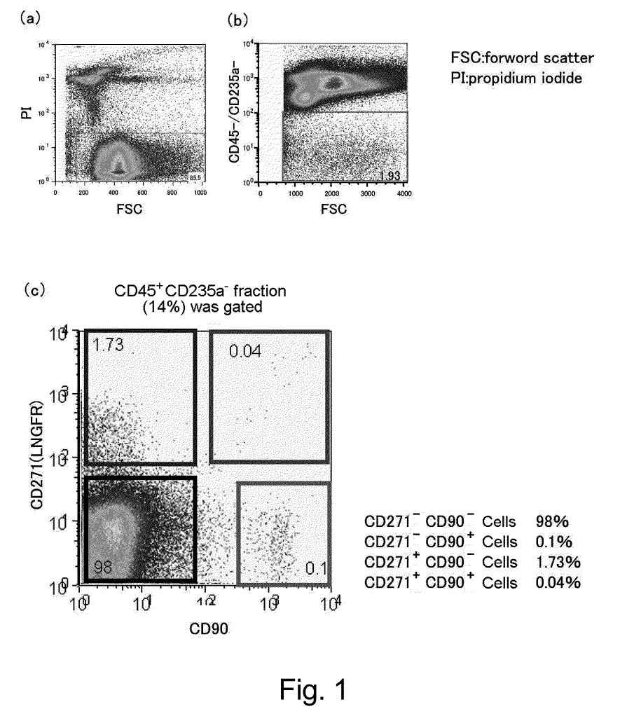

First, data from 1×10⁵ events were taken and PI-positive cells were gated out (FIG. 1(a)). Then, a CD45⁻CD235a⁻ fraction was gated (FIG. 1(b)). Finally, a fraction was gated, which was co-positive for CD90 (Thy-1) as the abscissa and for CD271 (LNGFR) as the ordinate. Afterwards, each of CD45⁻CD235a⁻CD271⁺CD90⁺ cells, CD45⁻CD235a⁻CD271⁺CD90⁻ cells, CD45⁻CD235a⁻CD271⁻CD90⁺ cells, CD45⁻CD235a⁻CD271⁻CD90⁻ cells, CD45⁻CD235a⁻CD271⁺ cells, CD45⁻CD235a⁻CD271⁻ cells, CD45⁻CD235a⁻CD90⁺ cells, and CD45⁻CD235a⁻CD90⁻ cells were fractionated and recovered. With respect to the numbers of non-blood cells, the CD45⁻CD235a⁻CD271⁺CD90⁺ cells, the CD45⁻CD235a⁻CD271⁺CD90⁻ cells, the CD45⁻CD235a⁻CD271⁻CD90⁺ cells and the CD45⁻CD235a⁻CD271⁻CD90⁻ cells accounted for 0.04%, 1.73%, 0.1%, and 98%, respectively (see FIG. 1(c)).

### Example 2

### Functional Analysis of CD45⁻CD235a⁻CD271⁺CD90⁺ Cells

### (1) Effect of Collagenase Treatment

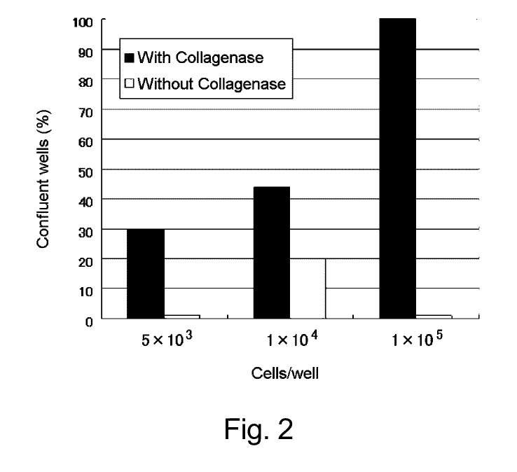

First, by using the cell suspensions obtained either with or without the collagenase treatment, the cells sorted into the CD45⁻CD235a⁻CD271⁺CD90⁺ fraction were suspended in a culture medium (DMEM: GIBCO 11885+20% FBS:Hyclone+bFGF+10 mM HEPES+1% P/S), from which 5×10³, 1×10⁴ and 1×10⁵ cells were seeded into respective wells of a 96-well culture dish and then cultured at 37° C. in a 5% CO₂ incubator. After 4 days, the culture supernatants were removed and fresh media were added; afterwards, the media were exchanged every 3 to 4 days. On Day 10, the numbers of the wells where cells became confluent were counted and their ratios were plotted as in FIG. 2.

As a result, it was found that if the bone marrow was treated with the collagenase, the CD45⁻CD235a⁻CD271⁺CD90⁺ cells could be recovered with higher yield at any cell densities.

### (2) Effect of Selection of CD271⁺CD90⁺Cells

Each of the cell fractions described in Example 1 was suspended in the medium (DMEM: GIBCO 11885+20% FBS:Hyclone+bFGF+10 mM HEPES+1% P/S), from which 100 to 8000 cells were seeded in 35 mm culture dishes, and incubated at 37° C. under 5% CO₂. After 4 days, the culture supernatants were removed and fresh growth media were added. The media were changed every 3 to 4 days. On Day 10, the culture dishes were observed under a phase-contrast microscope to count the number of colonies consisting of 50 cells or more. By calculating a ratio of the cells which formed colonies among the number of seeded cells, a frequency of the cells having CFU-F (fibroblast colony forming unit) activity was obtained, and compared as shown in the following table. It should be noted that WBM (whole bone marrow) means the total of the myelocytes.

TABLE 1
Comparison of CFU-F Frequencies among Cell Fractions

| WBM | CD90+ | CD271+ | CD90− | CD271− |
|---|---|---|---|---|
| 1/100000 | 8/4000 | 15/4000 | 0/300000 | 0/240000 |
| | | 15/4000 | 0/300000 | 0/240000 |

| CD271−CD90− | CD271−CD90+ | CD271+CD90− | CD271+CD90+ |
|---|---|---|---|
| 0/8000 | 0/8000 | 1/533 | 9/100 |
| | | | 10/100 |
| | | | 13/100 |

As shown in Table 1, the selection of the CD271⁺CD90⁺ cells could enrich the cells having CFU-F (fibroblast colony forming unit) activity by about 50 times and about 27 times more than the selection of CD90⁺ cells only and the selection of CD271⁺ cells only, respectively. In contrast, in neither of the selection of WBM, the selection of CD90⁻ cells only, nor the selection of CD271⁻ cells only, a cell having CFU-F activity was observed. In addition, a similar experiment as above was performed by seeding 100000 of WBM in a 100 mm culture dish, 300000 of CD90⁻ cells in a T75 culture flask, and 240000 of CD271⁻ cells in a T75 culture flask, but no cell having the CFU-F activity was observed either.

Further, by comparing the present results with data described in the literatures, the selection of the CD271⁺CD90⁺ cells were found to achieve the enrichment of the cells having the CFU-F activity much more efficiently than the selection of CD105⁺ cells only (Aslan H, et al., Stem Cells. 2006; vol. 24: p. 1728-1737) or the selection of CD271⁺ cells only (Quirici N et al. Exp Hematol. 2002; vol. 30: p. 783-791). It should be noted that the anti-CD105 antibody recognizes endothelial cells, early B lymphocytes and monocytes.

TABLE 2
Comparison of CFU-F Frequencies among Sepration Methods

| | | | CD271+ |
|---|---|---|---|
| WBM | CD105+ | CD271+ | CD90+ |
| 10⁵~10⁶* | 15873* | 120~5000* | 9~20 |

*Documented Values

### (2) Differentiation Assay

(i) Differentiation into Osteoblasts

### (a) Induction of Differentiation

The CD271⁺CD90⁺ cells after conducting the CFU-F assay were transferred to new plates; when they became confluent, the culture medium was changed from the growth medium to an osteoblast-inducing medium (CAMBREX PT-4120); and then the cells were incubated at 37° C. under 5% CO₂. The medium was changed to the fresh differentiation-inducing medium every 3 to 4 days and the differentiation was induced for two weeks.

### (b) Staining

The cells thus induced to differentiate were fixed with 4% PFA at room temperature for 10 min, and washed 3 times with PBS for 5 min. each. The osteoblasts were then stained with Histofine (Nichirei Biosciences, Code.415161), a kit of alkaline phosphatase (ALP) substrate.

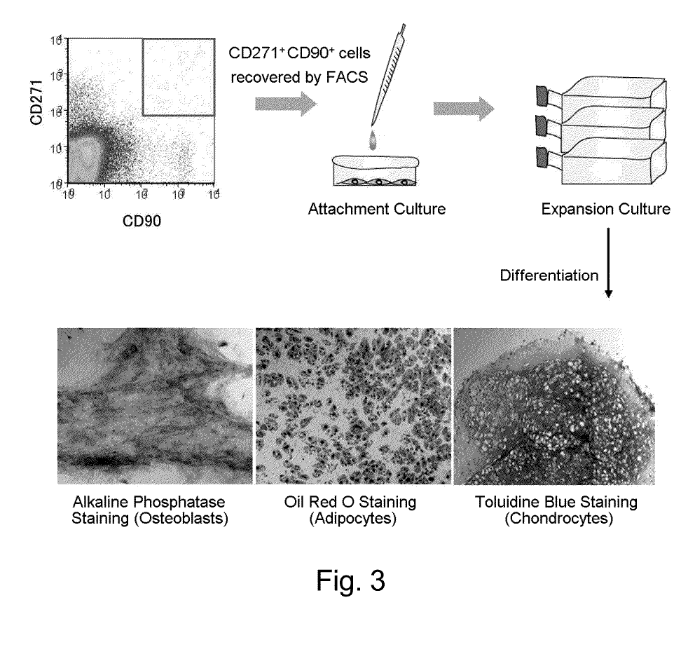

In a result as shown in FIG. 3, osteoblasts stained in pinkish to reddish colors (corresponding to gray to black colors in FIG. 3) were observed, indicating that the cells could differentiate into the osteoblasts.

### (ii) Adipocyte Differentiation Assay

### (a) Induction of Differentiation

The CD271⁺CD90⁺ cells remaining after the CFU-F assay were transferred to new plates; when they became confluent, the culture medium was changed from the growth medium to an adipocyte-inducing medium (CAMBREX PT-4135); and then the cells were incubated at 37° C. under 5% CO₂. After 3 days, the medium was changed to an adipocyte-maintaining medium (CAMBREX PT-4122), and such alternating exchanges of the medium between the adipocyte-inducing medium and the adipocyte-maintaining medium were repeated every 3 to 4 days and differentiation was induced for 2 weeks.

### (b) Staining

The cells thus induced to differentiate were fixed with 4% PFA at room temperature for 10 min, and washed 3 times with PBS for 5 min each. The adipocytes were then stained with Oil Red O Staining Solution (Muto Pure Chemicals, Lot No. 060822).

In a result as shown in FIG. 3, oil droplets of adipocytes stained in reddish colors (corresponding to gray to black colors in FIG. 3) were observed, indicating that the cells could differentiate into the adipocytes.

(iii) Chondrocyte Differentiation Assay

### (a) Induction of Differentiation

The CD271⁺CD90⁺ cells remaining after the CFU-F assay were transferred to new plates; when the cell number became 2×10⁵, the cells were suspended in a chondrocyte-inducing medium (CAMBREX PT-4121), transferred to 15 ml centrifuge tubes, and centrifuged for 5 min at ×150 g. After removing the supernatant, cells were resuspended in a chondrocyte-inducing medium supplemented with TGF-β3 (CAMBREX PT-4124) and BMP-6 (R&D Systems 507-BP/CF), and centrifuged for 5 min at ×150 g. The cells obtained in the form of a pellet were incubated as they are at 37° C. under 5% CO₂. The medium was changed to the fresh chondrocyte-inducing medium every 3 to 4 days and differentiation was induced for 3 weeks.

### (b) Staining

Clusters of the cells thus induced to differentiate were fixed with 4% PFA at room temperature for 1 hr, and washed 3 times with PBS for 5 min each. The cell clusters were paraffin-embedded and sliced into 6 μm sections. The sections were the stained with 0.05% toluidine blue solution (pH4.1, Wako 209-14545).

In a result as shown in FIG. 3, polysaccharides typical for chondrocytes stained in purplish colors (corresponding to gray to black colors in FIG. 3) were observed, indicating that the cells could differentiate into the chondrocytes.

To summarize, the CD45⁻CD235a⁻CD271⁺CD90⁺ cells are capable of differentiating into osteoblasts, chondrocytes and adipocytes, which are all mesenchymal cells. Thus, by selecting the CD45⁻CD235a⁻CD271⁺CD90⁺ cells from a cell population contained in a human bone marrow, human mesenchymal stem cells can be highly enriched.

### Example 3

### Exploration of Tissues where Mesenchymal Stem Cells are Present

This example demonstrates that the mesenchymal stem cells are present in a bone marrow, a peripheral blood, and a peripheral blood after an administration of G-CSF, but not in a cord blood.

A human cord blood, a peripheral blood, and a peripheral blood after an administration of G-CSF, obtained from specimens to be discarded from a patient, were used. From the human cord blood, the peripheral blood, and the peripheral blood after the administration of G-CSF, cell populations were prepared by following the method described in Example 1, and subjected to FACS analysis.

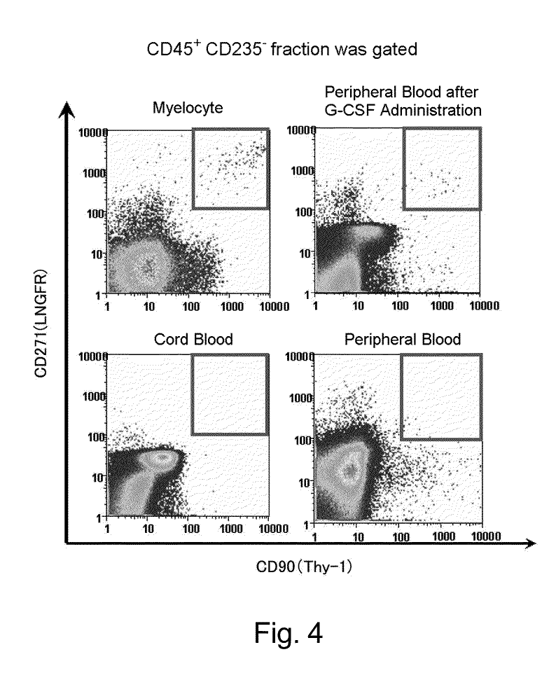

In a result as shown in FIG. 4, when a fraction negative for CD45 and negative for CD235a was gated, the obtained CD45⁻CD235a⁻CD271⁺CD90⁺ cells accounted for 0.01 to 0.04% in the bone marrow, 0 to 0.015% in the peripheral blood after the administration of G-CSF, and 0 to 0.008% in the peripheral blood. On the other hand, no CD45⁻CD235a⁻CD271⁺CD90⁺ cell was present in the cord blood.

### INDUSTRIAL APPLICABILITY

In accordance with the present invention, methods for highly enriching human mesenchymal stem cells from a cell population containing the human mesenchymal stem cells, as well as kits to be used therein can be provided.

1. A method for enriching human mesenchymal stem cells comprising the step of
selecting CD271⁺CD90⁺ cells expressing CD271 (LNGFR) and CD90 (Thy-1) from a cell population comprising the human mesenchymal stem cells.

2. The method according to claim 1, wherein the CD271⁺CD90⁺ cells are selected by using an anti-CD271 (LNGFR) antibody and an anti-CD90 (Thy-1) antibody.

3. The method according to claim 1, further comprising the step of preparing the cell population from a bone marrow.

4. The method according to claim 3, wherein the bone marrow is treated with collagenase in preparation of the cell population.

5. The method according to claim 1, further comprising the step of preparing the cell population from a peripheral blood after administration of G-CSF.

6. The method according to claim 1, further comprising the step of selecting CD45⁻CD235a⁻ cells that are not expressing CD45 nor CD235a.

7. The method according to claim 6, wherein the CD45⁻CD235a⁻ cells are selected by using an anti-CD45 antibody and an anti-CD235a antibody.

8. (canceled)

9. A kit comprising
an anti-CD271 antibody and
an anti-CD90 antibody.

10. The kit according to claim 9, further comprising
an anti-CD45 antibody and
an anti-CD235a antibody.

11. The kit according to claim 9, further comprising collagenase.
